(19) United States
(12) Patent Application Publication
(10) Pub. No.: US 2021/0002689 A1
(43) Pub. Date: Jan. 7, 2021

(54) METHOD FOR DETECTING DIFFERENT PROPERTIES IN A MICROORGANISM POPULATION BY OPTICALLY INDUCED DIELECTROPHORETIC FORCE

(71) Applicants: Jang-Jih Lu, Taipei City (TW); Min-Hsien Wu, Kaohsiung City (TW); Chih-Yu Chen, Taoyuan City (TW); Hsin-Yao Wang, Chiayi City (TW)

(72) Inventors: Jang-Jih Lu, Taipei City (TW); Min-Hsien Wu, Kaohsiung City (TW); Chih-Yu Chen, Taoyuan City (TW); Hsin-Yao Wang, Chiayi City (TW)

(73) Assignee: Chang Gung University, Taoyuan City (TW)

(21) Appl. No.: 16/779,776

(22) Filed: Feb. 3, 2020

(30) Foreign Application Priority Data
Jul. 2, 2019 (TW) 108123191

(51) Int. Cl. C12Q 1/04; B01L 3/00; C12M 1/34

(52) U.S. Cl. CPC C12Q 1/04; C12M 41/36; B01L 3/502715

(57) ABSTRACT

A method for detecting different properties in a microorganism population by ODEP force includes obtaining a microorganism sample solution having a plurality of microorganisms to be tested; pre-processing the microorganism sample solution to obtain a microorganism sample solution to be tested including the microorganisms having electrical properties differences; placing the microorganism sample solution to be tested in a channel of an ODEP device and activating an optical projection device to form at least one optical projection directed to the ODEP device; flowing the microorganism sample solution to be tested from one end of the channel to the other end thereof, and exerting an ODEP force on the optical projection device to generate a force having a direction different from a flowing direction of the microorganism sample solution to be tested; and detecting heterogeneity of the microorganisms based on strength differences of the ODEP force exerted on the respective microorganisms.

### BACKGROUND OF THE INVENTION

### 1. Field of the Invention

The invention relates to methods for detecting different properties in a microorganism population, and particularly to a method for detecting different properties in a microorganism population by optically induced dielectrophoretic (ODEP) force so as to conveniently, quickly provide a clinical access and report about heterogeneity of microorganisms, thereby increasing convenience, economics and correctness of healing.

### 2. Description of Related Art

Precision medicine can increase chances of healing. Precision medicine not only improves cancer medicine but also finds wide applications in dealing with epidemic. Antibiotics can heal specific pathogens in dealing with epidemic. However, in the current clinical diagnoses, an antibiotics susceptibility test (AST) conducted by a microorganism laboratory cannot provide a correct medical report. As such, a doctor cannot use antibiotics suggested by the medical report to cure a disease. Reason of the incorrect AST is that there is specific heterogeneity of microorganisms in samples.

While microorganism strains of the samples are the same, there are differences in genes, gene expression, protein expression, and metabolism among species of microorganisms. The differences mean that among microorganisms of the same species there are sub-species having different anti-medicine capabilities, different toxicities, and different viruses for causing diseases. Differences between the sub-species of the microorganisms are very small and they cannot be detected by the current clinical microorganism test methods. As a result, it is very difficult of finding correct antibiotics in clinical caring.

Currently, heterogeneity of microorganisms is found in many different important clinical species and they include Escherichia coli (E. coli), Staphylococcus aureus, Viridans streptococci, and Mycobacterium tuberculosis. For heterogeneous sub-species having high resistance to antibiotics, they can survive after being subjected to antibiotics. Next, the heterogeneous sub-species develop into larger groups by the selection of antibiotics. Therefore, heterogeneous sub-species having high resistance to antibiotics continuously grow and they pose a great challenge to the healthcare system throughout the world. Based on a report published by a Taiwan medical center, popularity of heterogeneous Vancomycin-intermediate Staphylococcus aureus (hVISA) increases from 0.7% in 2003 to 10.0% in 2013. For a single sample, heterogeneous resistance to antibiotics in microorganisms is a serious problem and it should be addressed correctly in clinical caring in the future.

People infected by pathogens having antibiotics heterogeneity have higher percentages of treatment failure or even death. The treatment failure can be attributed to ASTs conducted by most clinical microorganism laboratories cannot detect microorganism having heterogeneous resistance to antibiotics in a single sample. A doctor may refer to an AST report for prescribing correct antibiotics. However, the current ASTs cannot detect microorganism having heterogeneous resistance to antibiotics in the sample because the sub-species having heterogeneous resistance to antibiotics are a very small portion of the whole species such as in the range of 10⁻⁵ to 10⁻⁶. The current clinical microorganism test is conducted based on phenotype of microorganisms and a specific medicine being cultured. The phenotype is a result by means of macroscopic observation. Thus, it is impossible of detecting microscopic heterogeneity of the microorganisms. Currently, in research field of heterogeneity of microorganisms, a number of test methods are used and they include flow cytometry, brain heart infusion screening agar plates and population analysis profile—area under the curve (PAP-AUC). Next generation sequencing is also used to detect sub-species in sampled bacteria. However, these test methods are not widely used in clinical tests because they have drawbacks including being labor intensive, time consuming and cost ineffective. Therefore, they are not widely used in detecting heterogeneous sub-species in clinical tests.

Following are technical problems associated with the conventional art:

There is no technique being capable of systemically, conveniently and economically detecting heterogeneity of microorganisms in treating a patient in a clinical environment. The conventional technique is highly labor intensive. Further, cost is very high in detecting heterogeneity of microorganisms. Further, the whole cost is high enough to be practical. Therefore, it is not appropriate for research in laboratories and for detecting microorganism in a clinical environment.

The test may take several days or even weeks, i.e., being not time efficient. Thus, it does not satisfy the needs of clinical medicine and tracking subsequent progress.

Consequently, current clinical microorganism laboratories fail to detect a relatively minor heterogeneous microorganism population with high resistance or high toxicity in the clinical specimens. The inability of reporting heterogeneity of microorganism would cause incorrect diagnosis, treatment, and following-up in clinical practice.

Only a single target can be tested for heterogeneity and the target is nucleic acid, protein, membrane surface antigen, or metabolite. However, heterogeneity of microorganisms is not caused by a single factor. In fact, heterogeneity of microorganisms is caused by complicated interactions among molecules and factors. In other words, the conventional technique does not have the capability of testing heterogeneity of microorganisms.

Microorganisms are required to be processed prior to fixing a sample or decomposing bacteria in the conventional technique. However, the processed microorganisms are dead, thereby limiting possibilities of subsequent analysis and research.

Notwithstanding the conventional art, the invention is neither taught nor rendered obvious thereby.

### SUMMARY OF THE INVENTION

It is therefore one object of the invention to provide a method for detecting different properties in a microorganism population by ODEP force, comprising the steps of obtaining a microorganism sample solution having a plurality of microorganisms to be tested; pre-processing the microorganism sample solution to obtain a microorganism sample solution to be tested including the microorganisms having electrical properties differences; placing the microorganism sample solution to be tested in a channel of an ODEP device and activating an optical projection device to form at least one optical projection directed to the ODEP device; flowing the microorganism sample solution to be tested from one end of the channel to the other end thereof, and exerting an ODEP force on the optical projection device to generate a force having a direction different from a flowing direction of the microorganism sample solution to be tested; and detecting heterogeneity of the microorganisms based on strength differences of the ODEP force exerted on the respective microorganisms.

The invention has the following advantages and benefits in comparison with the conventional art:

Distant force may be generated in microorganisms by light, electricity or magnetism because heterogeneity in the microorganisms causes differences in conduction or polarizability. Further, flow control is employed to change the force for identifying heterogeneity in the microorganisms. Further, no damage of the microorganism is caused. Furthermore, the live microorganisms can be activated for separation. Its operation is convenient. Automation is made possible. Cost is greatly decreased. Clinical tests are made possible. A simple clinical test report of microorganisms can be made easily and is correct. It only takes several hours to make the test report. It fully satisfies the needs of clinical medication.

The above and other objects, features and advantages of the invention will become apparent from the following detailed description taken with the accompanying drawings.

### BRIEF DESCRIPTION OF THE DRAWINGS

FIG. 1 is a flow chart of a method for detecting different properties in a microorganism population by ODEP force according to the invention;

FIG. 2 schematically depicts projecting light from an optical projection device to an ODEP device;

FIG. 3 is an exploded view of the ODEP device;

FIG. 4 schematically depicts a microorganism sample solution to be tested being poured into the ODEP device;

FIG. 5 is a chart of ODEP force versus ampicillin concentration for heterogeneous E. coli having high resistance to antibiotics;

FIG. 6 is a chart of percentage of detected ATCC 35218 versus ratio of heterogeneity for rare E. coli having high resistance to antibiotics; and

FIG. 7 is a table of identified hVISA and VSSA in terms of clinically separated Staphylococcus aureus strains so as to evaluate performance of heterogeneity strains being resistant to antibiotics.

### DETAILED DESCRIPTION OF THE INVENTION

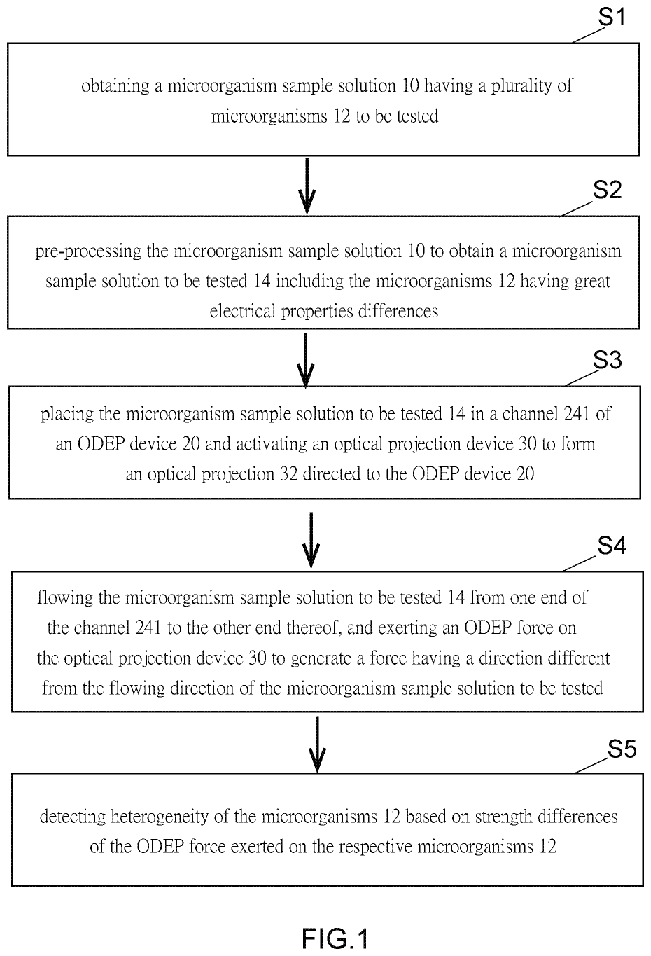

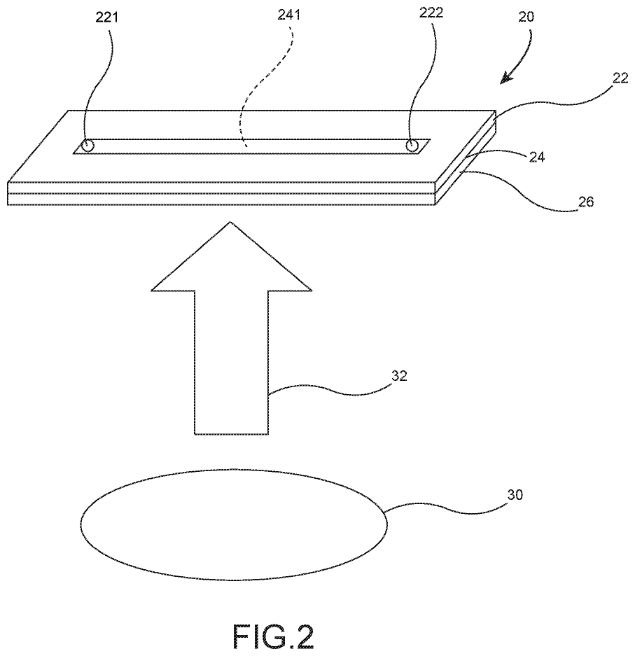

Referring to FIGS. 1 and 2, a method for detecting different properties in a microorganism population by ODEP force of the invention comprises the following steps:

S1: obtaining a microorganism sample solution 10 having a plurality of microorganisms 12 to be tested in which the microorganism sample solution 10 is comprised of cultured blood, urine, saliva, sweat, feces, pleural fluid, and ascites fluid (or cerebrospinal fluid).

S2: pre-processing the microorganism sample solution 10 to obtain a microorganism sample solution to be tested 14 including the microorganisms 12 having great electrical properties differences in which the pre-processing is microorganisms culturing, contact force, radiation, optical waves, acoustic waves, seismic waves, heating, refrigeration, electric waves, magnetic waves, drugs, or a combination thereof; and the electrical properties differences are caused by conduction or polarizability.

S3: placing the microorganism sample solution to be tested 14 in a channel 241 of an ODEP device 20 and activating an optical projection device 30 to form an optical projection 32 directed to the ODEP device 20.

S4: flowing the microorganism sample solution to be tested 14 from one end of the channel 241 to the other end thereof, and exerting an ODEP force on the optical projection device 30 to generate a force having a direction different from the flowing direction of the microorganism sample solution to be tested 14 in which the flowing direction of the microorganism sample solution to be tested 14 is controlled by contact force, gravity, electric force, magnetic force, heat, or a combination thereof.

S5: detecting heterogeneity of the microorganisms 12 based on strength differences of the ODEP force exerted on the respective microorganisms 12.

Preferably, features of the microorganisms 12 are species, sub-species, resistance to antibiotics, toxicity or metabolism.

Preferably, the microorganisms 12 are bacteria, molds, rickettsia species or viruses.

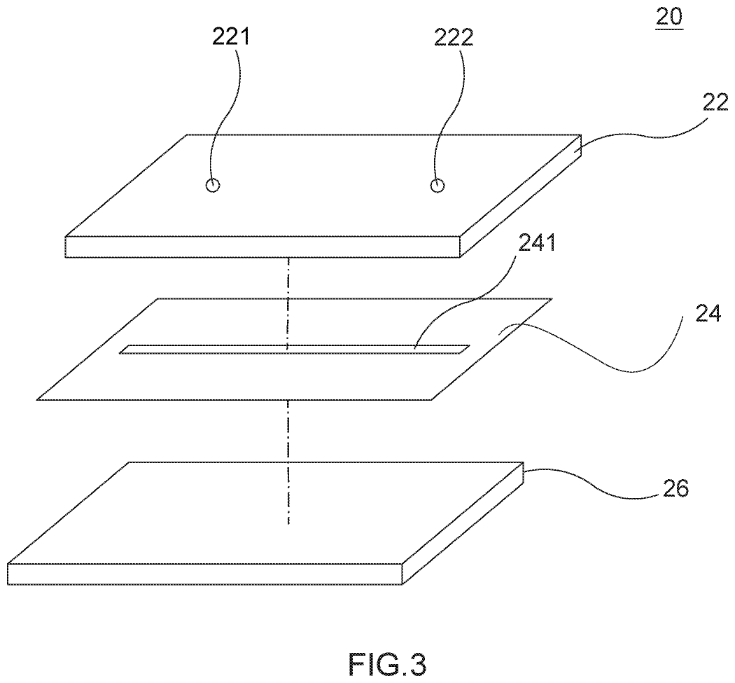

Referring to FIG. 3, the ODEP device 20 comprises, from top to bottom, a cover 22, a channel layer 24 and a photoconductive base 26. The cover 22 is formed of indium tin oxide (ITO), the channel layer 24 is formed of bio-compatible membrane, and the photoconductive base 26 is formed of photoconductive material. The channel 241 is formed on the channel layer 24. The cover 22 includes a sample injection hole 221 and a waste fluid collection hole 222 in which the sample injection hole 221 is aligned with one end of the channel 241 and the waste fluid collection hole 222 is aligned with the other end of the channel 241.

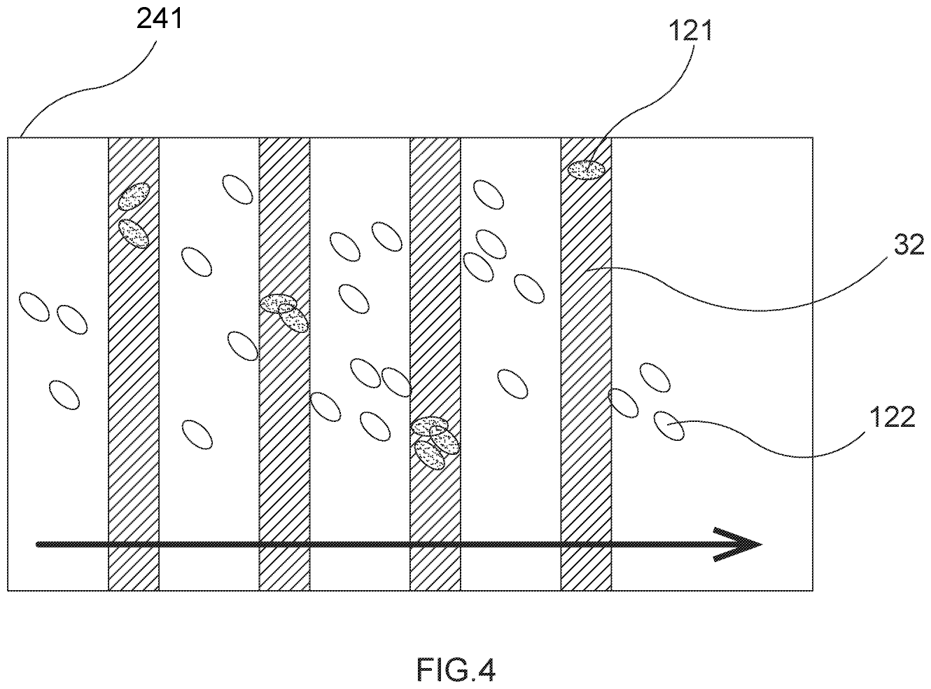

Referring to FIG. 4 in conjunction with FIGS. 1 to 3, it shows the microorganism sample solution to be tested 14 is poured into the channel 241 via the sample injection hole 221. The arrow indicates the flow direction of the microorganism sample solution to be tested 14. The microorganisms 12 include a plurality of first microorganisms 121 being high resistance to antibiotics and a plurality of second microorganisms 122 being low resistance to antibiotics. The microorganisms 12 generate great electrical properties differences. The first microorganisms 121 exert an opposite high ODEP force on the flow for capture and detection because they are high resistance to antibiotics and are highly polarizable. In response, a low ODEP force is exerted on the flow by the second microorganisms 122 because they are low resistance to antibiotics. But the second microorganisms 122 will not be intercepted and detected because they have low polarizability. Further, the second microorganisms 122 in the microorganism sample solution to be tested 14 flow to the waste fluid collection hole 222.

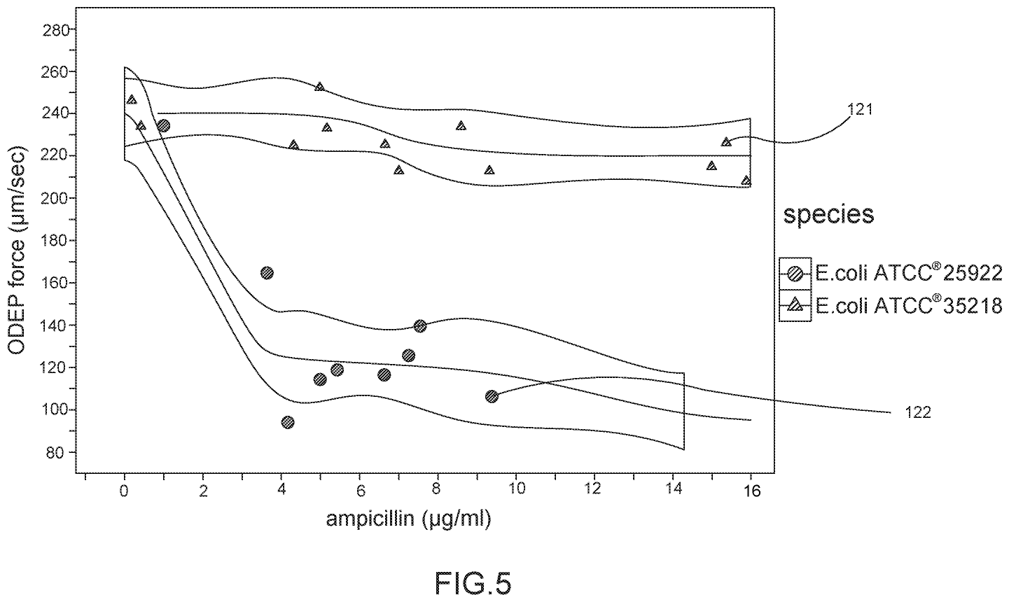

Referring to FIG. 5 in conjunction with FIGS. 1 to 4, Esherichia coli (E. coli) ATCC® 35218 and E. coli ATCC® 25922 having different properties of resistant to antibiotics ampicillin are used to establish a model of heterogeneity of microorganism resistant to antibiotics. The establishment of the model of heterogeneity of microorganism resistant to antibiotics and evaluation thereof are detailed below:

Culturing of E. coli:

E. coli ATCC® 35218 and E. coli ATCC® 25922 are widely used in clinical tests because their microorganisms have specific and stable properties. In terms of resistant to antibiotics, ampicillin of E. coli ATCC® 35218 has a minimal inhibition concentration greater than 32 μg/ml and ampicillin of E. coli ATCC® 25922 has a minimal inhibition concentration of 2-8 μg/ml. Both E. coli ATCC® 35218 and E. coli ATCC® 25922 are stored in −80° C. environment. In use, strains are taken from each of E. coli ATCC® 35218 and E. coli ATCC® 25922 and placed on two blood agar plates respectively. Next, the strains are cultured in 5% carbon dioxide concentration and 37° C. for 16 hours. The culturing is repeated for two generations in order to fully activate E. coli.

Subjecting E. coli to antibiotics ampicillin:

Referring to FIG. 1 again, after E. coli ATCC® 35218 and E. coli ATCC® 25922 have been activated, they are made into solutions having bacteria for subsequent antibiotics processing. Steps of making solutions having bacteria are described in detail below: selecting a plurality of activated large E. coli strains; dissolving it in normal saline which is a solution of sodium chloride at 0.9% concentration; adjusting its turbidity to 0.5 McFarland to make the microorganism sample solution 10; in each solution having bacteria, adding different amounts of ampicillin antibiotics powder to make a solution having antibiotics concentration of 0 μg/ml, 4 μg/ml, 8 μg/ml or 16 μg/ml, and finally, culturing each solution of different antibiotics concentration in 5% carbon dioxide concentration and 37° C. for 1.5 hours to make the microorganism sample solution to be tested 14.

Subjecting E. coli processed by antibiotics to ODEP force analysis:

Referring to FIGS. 1 to 4 again, an injection pump is used to inject the microorganism sample solution to be tested 14 into the ODEP device 20 via the sample injection hole 221. Electrical properties (e.g., conduction or polarizability) differences are generated because E. coli ATCC® 35218 and E. coli ATCC® 25922 in the microorganism sample solution to be tested 14 have been subjected to ampicillin. A plurality of optical projections 32 projected by the optical projection device 30 are interacted with the photoconductive base 26 of the ODEP device 20 to generate a different ODEP force exerted on a direction different from the flow direction of the microorganism sample solution to be tested 14. After subjecting E. coli ATCC® 35218 being highly resistant to antibiotics to ampicillin, E. coli ATCC® 35218 has significant electrical properties (e.g., conduction or polarizability). Thus, a high ODEP force is generated to cause the optical projections 32 to intercept and detect E. coli ATCC® 35218 in a direction opposite to the flow direction of the microorganism sample solution to be tested 14. To the contrary, after subjecting E. coli ATCC® 25922 being less resistant to antibiotics to ampicillin, E. coli ATCC® 25922 has insignificant electrical properties (e.g., conduction or polarizability). Thus, a low ODEP force is generated to not cause the optical projections 32 to intercept and detect E. coli ATCC® 25922 in the flow direction of the microorganism sample solution to be tested 14. Thus, E. coli ATCC® 25922 flows in the flow direction of the microorganism sample solution to be tested 14 to the waste fluid collection hole 222 of the ODEP device 20. Therefore, it is possible of determining differences between E. coli ATCC® 35218 and E. coli ATCC® 25922 which have been subjected to ampicillin of different concentrations in consideration of the optical projections 32 and the flow direction of the microorganism sample solution to be tested 14.

As shown in FIG. 5, since E. coli ATCC® 35218 has been subjected to ampicillin having a minimal inhibition concentration greater than 32 μg/ml, even after E. coli ATCC® 35218 has been subjected to ampicillin having a concentration of 0 μg/ml, 4 μg/ml, 8 μg/ml or 16 μg/ml, a force generated by light is still in the range of 220-260 μm/sec. To the contrary, after E. coli ATCC® 25922 has been subjected to ampicillin having a minimal inhibition concentration in the range of 2-8 μg/ml, in response to subjecting E. coli ATCC® 25922 to ampicillin gradually having an increased concentration, a force generated by light is decreased to 100 μm/sec.

In brief, after E. coli ATCC® 35218 and E. coli ATCC® 25922 have been subjected to antibiotics of different concentrations, E. coli ATCC® 35218 strains being highly resistant to antibiotics are intact. To the contrary, after E. coli ATCC® 25922 being less resistant to antibiotics has been subjected to ampicillin having a minimal concentration higher than a minimal inhibition concentration, E. coli ATCC® 25922 strains are damaged and electrical properties of the cells thereof are changed. In view of above analysis, the method of the invention can identify microorganisms 12 being different in resistance to antibiotics.

Collection ratio of E. coli ATCC® 35218 strains resistant to antibiotics under different ratios of heterogeneity of E. coli:

Following is an embodiment for describing the method of the invention is still capable of detecting rare strains being highly resistant to antibiotics in a microorganism population when heterogeneity of microorganisms is less significant to resistance of antibiotics.

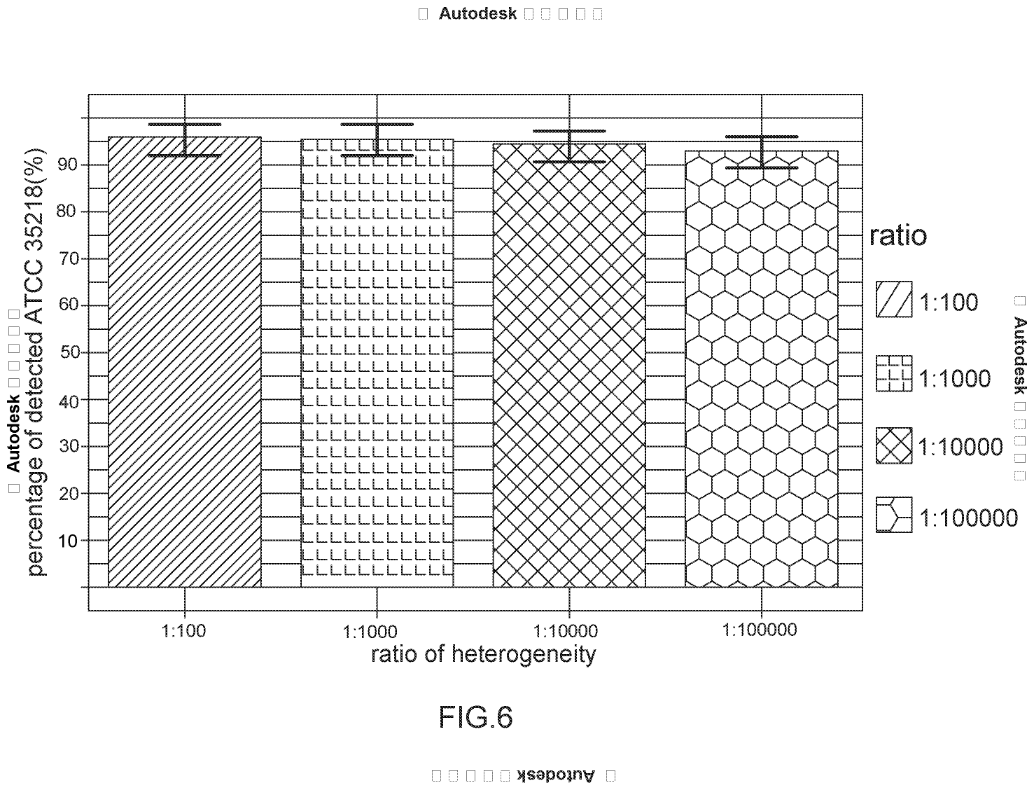

Referring to FIG. 6 in conjunction with FIGS. 1 to 4, in the embodiment of simulating a disease in a clinical environment, possible heterogeneity ratios in a microorganism population is implemented by mixing different ratios of E. coli ATCC® 35218 strains and E. coli ATCC® 25922 strains in which E. coli ATCC® 35218 and E. coli ATCC® 25922 are mixed in ratios of 1:100, 1:1000, 1:10000, or 1:100000. Regarding the above mixed microorganism solution, its concentration is adjusted to 0.5 McFarland (1.5×10⁸ CFU/ml) and the solution is mixed at a ratio of each of 1:100, 1:1000 and 1:10000. Next, the solution is dissolved in normal saline which is a solution of sodium chloride at 0.9% concentration and the volume ratios of the solution to the saline is 1000, 100 and 10 respectively. As a result, the amount of E. coli ATCC® 35218 in the solution in each ratio is 1000 CFU. For ratios of E. coli ATCC® 35218 (having strains being highly resistant to antibiotics) to E. coli ATCC® 25922 (having strains being less resistant to antibiotics) being 1:100, 1:1000, 1:10000 or 1:100000, the method of the invention can identify E. coli ATCC® 35218 (having strains being highly resistant to antibiotics) at a success rate more than 95%. It is concluded that the method of the invention is still capable of detecting rare strains being highly resistant to antibiotics in a microorganism population when heterogeneity of microorganisms is less significant to resistance of antibiotics.

Confirmation of effect of the invention applying to heterogeneity of microorganism resistant to antibiotics in a clinical environment:

Culturing of Staphylococcus aureus and confirmation of heterogeneous resistance to antibiotics both in a clinical environment:

For confirmation of effect of the invention applying to heterogeneity of microorganism resistant to antibiotics in a clinical environment, culturing of Staphylococcus aureus and confirmation of heterogeneous resistance to antibiotics both in a clinical environment are done. Currently, hVISA is clinically the most important microorganism having heterogeneous resistance to antibiotics. The characteristic of hVISA resistant to antibiotics cannot be detected clinically by AST. hVISA is erroneously identified as vancomycin-susceptible Staphylococcus aureus (VSSA) in view of expression. For correctly distinguishing hVISA from VSSA, a modified population analysis profile—area under the curve (PAP-AUC) should be taken for analysis. As a result, hVISA is correctly identified.

Sources of the microorganism sample solutions 10 are from wards of Chang Gung Memorial Hospital in Linkou, Taiwan. The microorganism sample solutions 10 are sent to a microorganism laboratory to culture into many different microorganism sample solutions including blood, urine, saliva, sweat, feces, pleural fluid, ascites fluid and cerebrospinal fluid. Firstly, the microorganism sample solution 10 is cultured on a blood agar plate. Next, the microorganism sample solution 10 is cultured in 5% carbon dioxide concentration and 37° C. for 16-18 hours. After being cultured, a single strain group is taken from the blood agar plate and is subjected to a matrix-assisted laser desorption ionization time-of-flight (MALDI-TOF) for strains identification. With respect to selecting strains to be identified as Staphylococcus aureus strains, Vancomycin Etest is used in which 125 strains having a minimal inhibition concentration of 2-4 μg/mL are selected for PAP-AUC. Finally, the 125 Staphylococcus aureus strains are identified as 35 hVISA strains and 90 VSSA strains.

Subjecting Staphylococcus aureus to antibiotics Vancomycin:

After 35 hVISA strains and 90 VSSA strains have been activated, they are made into solutions having bacteria for being subjected to antibiotics thereafter. The following steps are performed to make a desired solution: selecting a number of activated Staphylococcus aureus strains; dissolving it in normal saline which is a solution of sodium chloride at 0.9% concentration; adjusting its turbidity to 0.5 McFarland; adding Vancomycin antibiotics powder in the solution having bacteria to have antibiotics at 4 μg/ml concentration, and culturing it in 5% carbon dioxide concentration and 37° C. for 2 hours to make the microorganism sample solution to be tested 14.

Using the method of the invention to analyze Staphylococcus aureus which has been subjected to antibiotics and evaluating the performance:

Clinically, the resistance to antibiotics of hVISA and VSSA strains are confirmed by PAP-AUC. Polarizability of the strains is changed because the resistance to antibiotics has been change after has been subjected to antibiotics. By utilizing the change and subjecting 35 hVISA strains and 90 VSSA strains to be analyzed by the method of the invention, Staphylococcus aureus strains are separated clinically and identified as heterogeneity strains being resistant to antibiotics.

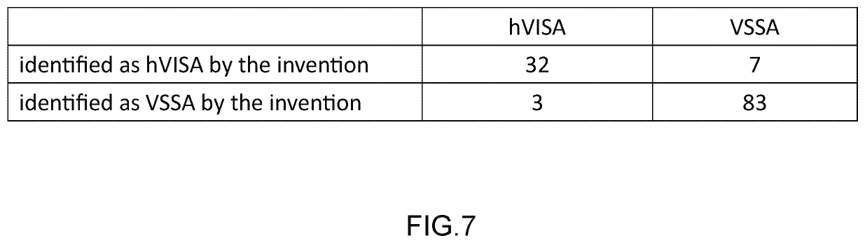

Referring to FIG. 7, its result is shown. 35 strains are identified as hVISA strains by PAP-AUC. 32 strains are detected. Sensitivity of the detection is 91.42% (32/35). Also, 90 strains are identified as VSSA strains by PAP-AUC. 83 strains are detected. Specificity of the detection is 92.22% (83/90). After being tested clinically by the method of the invention, both sensitivity and specificity are more than 90% and taken time is about one tenth of PAP-AUC. It is concluded that the invention is highly applicable.

The invention has the following advantages and benefits in comparison with the conventional art: after subjecting heterogeneity of microorganisms 12 to antibiotics, ODEP force is employed and flow control is performed so that a medical employee may quickly, conveniently test heterogeneity in a microorganism sample solution in a clinical setting. Further, a correct test report about heterogeneity of microorganisms 12 can be made and provided to a licensed healthcare professional or doctor, thereby increasing convenience, economics and correctness of healing; making phenotype analysis easier; and being more effective in clinical practice.

Cost of materials employed by ODEP force is much less than that employed by the conventional molecular test. Regarding time, the Invention only takes several hours to finish the analysis of heterogeneity in microorganisms 12. Thanks to ODEP force and the flow control, microorganisms 12 can be kept viable by the method of the invention. This facilitates subsequent analyses. It is concluded that the method of the invention is appropriate to clinically test microorganisms 12.

While the invention has been described in terms of preferred embodiments, those skilled in the art will recognize that the invention can be practiced with modifications within the spirit and scope of the appended claims.

What is claimed is:

1. A method for detecting different properties in a microorganism population by ODEP force, comprising the steps of:
(a) obtaining a microorganism sample solution having a plurality of microorganisms to be tested;
(b) pre-processing the microorganism sample solution to obtain a microorganism sample solution to be tested including the microorganisms having electrical properties differences;
(c) placing the microorganism sample solution to be tested in a channel of an ODEP device and activating an optical projection device to form at least one optical projection directed to the ODEP device;
(d) flowing the microorganism sample solution to be tested from one end of the channel to the other end thereof, and exerting an ODEP force on the optical projection device to generate a force having a direction different from a flowing direction of the microorganism sample solution to be tested; and
(e) detecting heterogeneity of properties of the microorganisms based on strength differences of the ODEP force exerted on the respective microorganisms.

2. The method of claim 1, wherein the ODEP device comprises, from top to bottom, a cover, a channel layer, and a photoconductive base; wherein the channel is formed on the channel layer; wherein the cover includes a sample injection hole and a waste fluid collection hole; and wherein the sample injection hole is aligned with one end of the channel and the waste fluid collection hole is aligned with the other end of the channel.

3. The method of claim 2, wherein the cover is formed of ITO and the channel layer is formed of bio-compatible membrane.

4. The method of claim 1, wherein the pre-processing is microorganisms culturing, contact force, radiation, optical waves, acoustic waves, seismic waves, heating, refrigeration, electric waves, magnetic waves, drugs, or a combination thereof.

5. The method of claim 1, wherein the flowing direction of the microorganism sample solution to be tested is controlled by contact force, gravity, electric force, magnetic force, heat, or a combination thereof.

6. The method of claim 1, wherein the electrical properties differences are caused by conduction or polarizability.

7. The method of claim 1, wherein features of the microorganisms are species, sub-species, resistance to antibiotics, toxicity, or metabolism.

8. The method of claim 1, wherein the microorganisms are bacteria, molds, rickettsia species or viruses.

9. The method of claim 1, wherein the microorganism sample solution includes blood, urine, saliva, sweat, feces, pleural fluid, ascites fluid, and cerebrospinal fluid.
